(19) United States
(12) Patent Application Publication
(10) Pub. No.: US 2014/0023721 A1
(43) Pub. Date: Jan. 23, 2014

(54) METHOD OF EXTRACTING ACTIVE MOLECULES FROM NATURAL RESINS AND USE THEREOF

(71) Applicants: Luigi Amelotti, Busto Arsizio (IT); Lorenzo Secondini, Busto Arsizio (IT)

(72) Inventors: Luigi Amelotti, Busto Arsizio (IT); Lorenzo Secondini, Busto Arsizio (IT)

(73) Assignee: RODE PHARMA S.R.L., Busto Arsizio (IT)

(21) Appl. No.: 13/992,978

(22) PCT Filed: Dec. 9, 2011
(86) PCT No.: PCT/IB2011/003013

§ 371 Date: Sep. 3, 2013

(30) Foreign Application Priority Data
Dec. 10, 2010 (IT) MI2010A002268

(51) Int. Cl. A61K 36/45; A23L 1/30; A61K 35/64; A61K 36/328; A61K 36/28

(52) U.S. Cl. CPC A61K 36/45; A61K 36/328; A61K 36/28; A61K 35/644; A23L 1/3002
USPC 424/537; 424/748; 424/764; 424/777

(57) ABSTRACT

The present invention relates to a method of extracting active molecules from natural resins and/or essential oils. In particular, the present invention relates to a method of extracting active molecules selected from the group comprising terpenes, flavonoids, anthocyanins and catechins. Moreover, the present invention relates to an extract, preferably in liquid form, obtained with said method. Finally, the present invention relates to the use of said extract containing the active molecules selected from the group comprising terpenes, flavonoids, anthocyanins and catechins for the preparation of a food composition or supplement or a pharmaceutical composition.

The present invention relates to a method of extracting active molecules from a vegetable substrate, natural resins and/or essential oils. In particular, the present invention relates to a method of extracting active molecules selected from the group comprising terpenes, flavonoids, anthocyans and catechins.

Furthermore, the present invention relates to an extract, preferably in liquid form, obtained with said method. Finally, the present invention relates to the use of said extract containing the active molecules selected from the group comprising terpenes, flavonoids, anthocyans and catechins for the preparation of a food composition or a supplement product or a pharmaceutical composition.

It is known that natural resins such as, for example, myrrh, incense and propolis contain large quantities of active molecules such as, for example, terpenes and/or flavonoids. For example, monoterpenes (2 isoprene units and 10 carbon atoms), sesquiterpenes (3 isoprene units and 15 carbon atoms) or triterpenes (6 isoprene units and 30 carbon atoms) can be found. For example, quercetin and epicatechin can be found.

In the above-mentioned natural resins, present together with the above-mentioned active molecules there are also sugars, starches, gums and other polymeric components which bind to the active molecules, limiting the extraction thereof.

It is known that one method for extracting the active molecules contained in natural resins is represented by a method of extraction by steam distillation.

However, the method of extraction by steam distillation presents many limits and drawbacks which limit its use, such as, for example, a low extraction yield associated with a limited number of molecules extracted. Several extraction methods that use gases such as CO₂ or N₂ under supercritical conditions (supercritical gases) are also known. For example, carbon dioxide becomes supercritical at a temperature of 31° C. and pressure of 73 atmospheres.

Compared to methods of extraction by steam distillation, said methods of extraction with supercritical CO₂ or N₂ enable a larger number of active molecules to be extracted and with a larger yield from a quantitative viewpoint.

However, said extraction methods which use supercritical gases such as CO₂ or N₂ present several limits and drawbacks which limit their use.

One limit is given by the costs of constructing the equipment and of maintaining it.

Another limit is given by the fact that, under the extraction operating conditions, undesired reaction products (e.g. molecular aggregates and/or by-products) are generated as a result of cross reactions between the molecules themselves. For these reasons, the method of extraction by steam distillation today still remains the most widely used extraction method.

However, a great limit presented by the method of extraction by steam distillation is the fact that in order to increase the extraction efficiency, the number of molecules extracted, the percentage of extraction and the extraction yield it is necessary to operate within particular operating conditions, avoiding temperatures, solvent mixtures and pressure values that could damage the chemical and/or physical nature of the active molecules extracted, which would lose their functional activity and, as a consequence, their commercial interest as active molecules functional for body.

For example, an extraction method that operates at an extraction temperature greater than 100° C. can damage (denature) the themosensitive active molecules extracted.

For example, almost the totality of flavonoids degrades in a temperature interval of 52° C. to 85° C.

Patent EP 1641903 B1 relates to a method for the extraction of terpenes and/or terpenoids from natural resins using polar solvents in the presence of a rotating magnetic field.

However, said method of extraction with a rotating magnetic field presents several limits and drawbacks which limit its use.

One limit is given by the costs of constructing the equipment and of maintaining it. In particular, the operation of the rotating magnetic field requires highly sophisticated magnetic field control devices to ensure the correct magnetic field interval. Another limit is given by the fact that it is necessary to operate with a magnetic field comprised from 1000 to 3500 Gauss. Moreover, the extraction capacity depends on the size of the generator that creates the rotating magnetic field. Therefore, in order to generate magnetic fields comprised from 1000 to 3500 Gauss, it is necessary to have large apparatus with large magnets that need to be shielded in an appropriate manner.

Therefore, there remains a need to have a method of extracting active molecules from a vegetable substrate and/or natural resins and/or essential oils that does not present the limits and drawbacks of the known methods.

In particular, there remains a need to have a method of extracting active molecules from a vegetable substrate and/or natural resins and/or essential oils that is simple, economical, easy to manage and practical, while at the same time guaranteeing a high extraction efficiency, understood as the number of extracted active molecules, and a high extraction yield, understood as the quantity by weight of the extracted molecules.

In particular, there remains a need to have a method of extracting active molecules from a vegetable substrate and/or natural resins and/or essential oils that is capable of extracting a large number of molecules in large quantities and which, under the operating conditions, is capable of maintaining intact the chemical and/or physical structure of the extracted active molecules. Practically speaking, there is a felt need to have an extraction method which is capable of avoiding the chemical and/or physical degradation/decomposition of the extracted molecules or a modification/loss of the original chemical structure with a consequent loss of commercial value as active molecules that are functional for the body. Therefore, the present invention relates to a method of extracting molecules from a vegetable substrate and/or natural resins and/or essential oils, having the characteristics set forth in the appended claim.

Moreover, the present invention relates to an extract containing active molecules, extracted from a vegetable substrate and/or natural resins and/or essential oils, having the characteristics set forth in the appended claim.

Finally, the present invention relates to the use of said extract of active molecules for the preparation of a food composition or supplement product or pharmaceutical composition, having the characteristics set forth in the appended claim.

Finally, the present invention relates to an apparatus for carrying out said extraction method, having the characteristics set forth in the appended claim.

Some preferred embodiments of the present invention are set forth in the detailed description that follows, without intending in any way to limit the scope of the present invention.

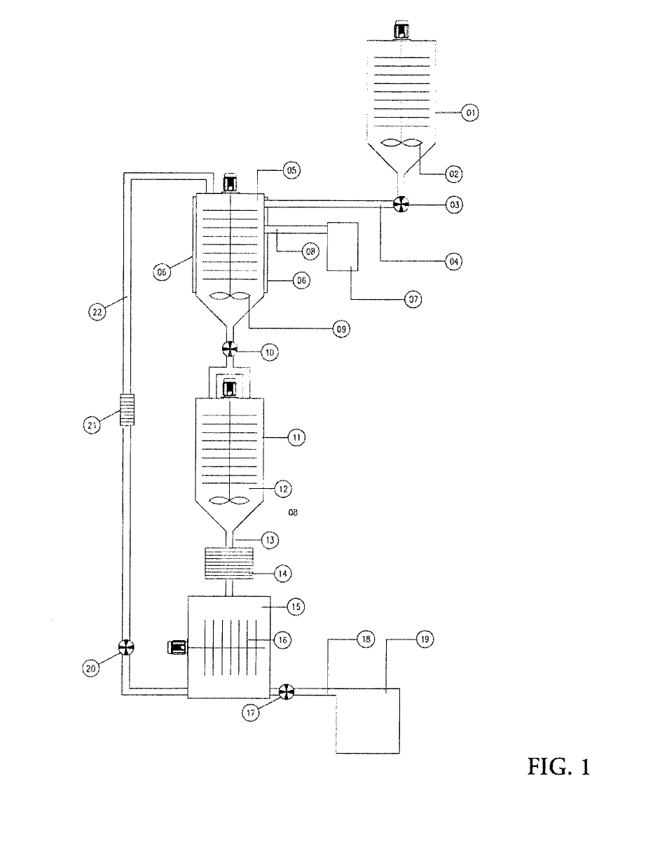

FIG. 1 relates to an apparatus for carrying out the extraction method of the present invention.

Table 1 refers to the qualitative and quantitative analyses conducted on the extract obtained with the extraction method of the present invention when carried out on myrrh, as per example 1.

Table 2 refers to the qualitative and quantitative analyses conducted on the extract obtained with the extraction method of the present invention when carried out on incense, as per example 2.

Table 3 refers to the qualitative and quantitative analyses conducted on the extract obtained with the extraction method of the present invention when carried out on Tanacetum Parthenium, as per example 3.

Table 4 refers to the qualitative and quantitative analyses conducted on the extract obtained with the extraction method of the present invention when carried out on cranberry, as per example 4.

Table 5 refers to the qualitative and quantitative analyses conducted on the extract obtained with the extraction method of the present invention when carried out on propolis, as per example 5.

Table 6 refers to the qualitative and quantitative analyses conducted on the extract obtained with the extraction method of the present invention when carried out on cranberry, as per example 6.

Table 7 refers to the qualitative and quantitative analyses conducted on the extract obtained with the extraction method of the present invention when carried out on Tanacetum Parthenium, as per example 7.

Table 8 refers to the qualitative and quantitative analyses conducted on the extract obtained with the extraction method of the present invention when carried out on incense, as per example 8.

Table 9 refers to the qualitative and quantitative analyses conducted on the extract obtained with the extraction method of the present invention when carried out on myrrh, as per example 9.

Table 10 refers to the qualitative and quantitative analyses conducted on the extract obtained with the extraction method of the present invention when carried out on propolis, as per example 10.

The Applicant has conceived a new method of extracting active molecules that are naturally present in a vegetable substrate and/or natural resins and/or essential oils. The method comprises at least a step in which said vegetable substrate is placed in contact with an extraction liquid, said extraction liquid being obtained by adding an extraction gas (as described below) in the gaseous state to a liquid solvent (as described below) selected from the group comprising polar solvents and/or non-polar solvents.

The extraction method of the present invention envisages the use of an extraction liquid. The extraction liquid comprises or, alternatively, consists of an extraction solvent (A) and an extraction gas (Y).

The extraction gas (Y) is represented by any substance which, at a temperature of 23° C. and pressure of 1 atmosphere, is in a gaseous state. Gases brought into supercritical conditions (so-called supercritical gases) are not contemplated in the context of the present invention. For example, supercritical carbon dioxide does not have valid application in the context of the present invention.

Said extraction gas (Y) is selected from the group comprising or, alternatively, consisting of helium, neon, argon, krypton, xenon, carbon dioxide, nitrogen and oxygen or mixtures thereof.

Advantageously, said extraction gas is selected from the group comprising argon, nitrogen, carbon dioxide or mixtures thereof.

The extraction gas (Y) is present in an amount comprised from 0.1 to 10% by volume, relative to100 parts by volume of extraction solvent used.

Advantageously, the extraction gas is present in an amount comprised from 0.5 to 5% by volume, preferably from 1 to 2.5% by volume, relative to100 parts by volume of extraction solvent used.

Advantageously, the extraction gas comprises carbon dioxide CO₂, which at a temperature of 23° C. and pressure of 1 atmosphere is in the gaseous state. In a preferred embodiment, the extraction gas consists of carbon dioxide CO₂ (Y1). The extraction gas comprising or, alternatively, consisting of carbon dioxide, is present in the extraction liquid in an amount comprised from 0.1 to 5%, preferably 2.5 or 3%, by volume, relative to 100 parts by volume of extraction solvent used.

Advantageously, the extraction gas comprises argon, which at a temperature of 23° C. and pressure of 1 atmosphere is in the gaseous state. In a preferred embodiment, the extraction gas consists of argon (Y2). The extraction gas comprising or, alternatively, consisting of argon, is present in the extraction liquid in an amount comprised from 0.1 to 5%, preferably 2.5 or 3%, by volume, relative to 100 parts by volume of extraction solvent used.

Advantageously, the extraction gas comprises nitrogen, which at a temperature of 23° C. and pressure of 1 atmosphere is in the gaseous state. In a preferred embodiment, the extraction gas consists of nitrogen (Y3). The extraction gas comprising or, alternatively, consisting of nitrogen, is present in the extraction liquid in an amount comprised from 0.1 to 5%, preferably 2.5 or 3%, by volume, relative to100 parts by volume of extraction solvent used.

Advantageously, the extraction gas comprises argon and carbon dioxide, which at a temperature of 23° C. and pressure of 1 atmosphere are in the gaseous state. In a preferred embodiment, the extraction gas consists of argon and carbon dioxide (Y4). The extraction gas comprising or, alternatively, consisting of a argon and carbon dioxide mixture, is present in the extraction liquid preferably in a ratio comprised from 1:3 to 3:1, 1:1; or in an amount comprised from 0.1 to 5%, preferably 2.5 or 3%, by volume, relative to 100 parts by volume of extraction solvent used.

Advantageously, the extraction gas comprises argon and nitrogen, which at a temperature of 23° C. and pressure of 1 atmosphere are in the gaseous state. In a preferred embodiment, the extraction gas consists of argon and nitrogen (Y5). The extraction gas comprising or, alternatively, consisting of an argon and nitrogen mixture, is present in the extraction liquid preferably in a ratio comprised from 1:3 to 3:1, 1:1; or in an amount comprised from 0.1 to 5%, preferably 2.5 or 3%, by volume, relative to 100 parts by volume of extraction solvent used.

Advantageously, the extraction gas comprises nitrogen, argon and carbon dioxide, which, at a temperature of 23° C. and pressure of 1 atmosphere are in the gaseous state. In a preferred embodiment, the extraction gas consists of nitrogen, argon and carbon dioxide (Y6). The extraction gas comprising or, alternatively, consisting of a nitrogen, argon and carbon dioxide mixture, is present in the extraction liquid preferably in a ratio comprised from 1:3:1 to 1:1:1 (N₂:Ar₂:CO₂); or in an amount comprised from 0.1 to 5%, preferably 2.5 or 3%, by volume, relative to100 parts by volume of extraction solvent used.

The extraction solvent (A), in liquid form, comprises one or more compounds, as illustrated below.

The extraction solvent in liquid form comprises or, alternatively, consists of a polar solvent or a mixture of polar solvents, or a non-polar solvent or a mixture of non-polar solvents, or a mixture of polar solvents and non-polar solvents (Group A1). Said extraction solvent may be of an aliphatic and/or aromatic nature (Group A1).

In the context of the present invention, the solvents are classified into two categories based on the value of the dielectric constant: polar solvents and non-polar solvents. Water has a dielectric constant value of about 80 to 20° C. (polar solvent), whereas solvents having a dielectric constant values of less than 15 are generally classified as non-polar. Polar solvents can be divided into protic polar solvents and aprotic polar solvents.

In a preferred embodiment, the extraction solvent comprises at least one compound having at least a carboxyl group and/or ester group (Group B1). Said compound, when it comprises at least a carboxyl group (and does not contain an ester group) is selected from the group comprising, or consisting of, at least a monocarboxylic, dicarboxylic, tricarboxylic and tetracarboxylic compound. The carboxyl group can also be present in protected form, in the form of an ester (Group B2).

In a preferred embodiment, the extraction solvent comprises at least one aliphatic monocarboxylic compound having the formula (I) [R—COOH], wherein R represents: a C1-C10 alkyl group, preferably C1-C5; an R1-CH₂(OH)— group, where R1 has the same meaning as R (Group B3). Preferably, the acid used is formic acid or propionic acid or mixtures thereof.

In the context of the present invention, the extraction solvent (A) comprises or, alternatively, consists of acetic acid. The acetic acid can be 6 molar acetic acid, 12 molar acetic acid or glacial acetic acid with a purity of at least al 95%, preferably 98%. The extraction solvent comprises or, alternatively, consists of a solution of acetic acid and water. Preferably, when the acid is acetic acid, it is used in a 12% aqueous solution.

Advantageously, the extraction liquid comprises or, alternatively, consists of an extraction solvent (A) which comprises or, alternatively, consists of acetic acid or a solution of acetic acid and water, and of an extraction gas selected from the group comprising or, alternatively, consisting of Y1, Y2, Y3, Y4, Y5 and Y6.

In a preferred embodiment, the extraction solvent comprises at least one aliphatic dicarboxylic compound having the formula (II) [HOOC—(CₙH₂ₙ₊₂)—COOH], wherein “n” can be equal to zero or can be comprised from 1 to 10, preferably n=0 (Group B4). Advantageously, the extraction solvent comprises oxalic acid or malonic acid or mixtures thereof.

In a preferred embodiment, the extraction solvent has an aromatic chemical structure and comprises at least one carboxyl group (Group B5).

Advantageously, the extraction solvent comprises benzoic acid (Ph-COOH) or benzoic acid substituted in the ortho, meta or para position with an aliphatic alkyl group R, having the formula (III)[R-Ph-COOH], meaning a C1-C 4 short-chain alkyl.

Advantageously, the extraction solvent comprises benzoic acid substituted with a methyl group in the ortho position.

In a preferred embodiment, the extraction solvent comprises at least one tricarboxylic compound (Group B6). Advantageously, the extraction solvent comprises citric acid.

In a preferred embodiment, the extraction solvent comprises at least one tetracarboxylic compound (Group B7). Advantageously, the solvent comprises pyromellitic acid (CAS 89-05-4).

As mentioned above, the extraction solvent can comprise at least one compound having at least a carboxyl group and/or an ester group (Group B1).

In the event that said solvent comprises at least one compound having at least an ester group (and no carboxyl group), said compound is selected from the group comprising the esters having the formula (IV) R—C(O)O—R1, where the group R can be equal to R1 or different from R1; the groups R and R1 can be C1-C5 short-chain alkyl groups. Preferably, R is a methyl group and R1 is a methyl, ethyl or propyl group (Group B8).

In a preferred embodiment, the extraction solvent (A), when it comprises at least one compound having at least a carboxyl group and at least an alcohol group, has the formula (VII) [R—CH(OH)—COOH], where R is selected from among the C1-C4 short-chain aliphatic groups (Group B9). Advantageously, R is methyl.

All of the above-defined compounds (Groups B2-B9) belong to the group of compounds B1.

In a preferred embodiment, the extraction solvent (A) comprises at least one compound having at least an alcohol group (Group C1)

Said compound having at least an alcohol group comprises, or alternatively consists of, a primary, secondary or tertiary aliphatic alcohol (Group C2).

In a preferred embodiment, the extraction solvent comprises at least a primary aliphatic alcohol having the formula (V) R—OH, wherein R represents: a C1-C10 alkyl group, preferably C1-C5 (Group C3).

Advantageously, the alcohol is selected from the group comprising ethanol, hexanol and octanol.

In one embodiment of the present invention, the extraction solvent comprises or, alternatively, consists of acetic acid and ethyl alcohol, in a ratio comprised from 1:2 to 2:1; preferably it is an aqueous solution of acetic acid and ethyl alcohol.

The extraction liquid can comprise or, alternatively, consist of acetic acid and ethyl alcohol, or an aqueous solution of acetic acid and ethyl alcohol, and of an extraction gas selected from the group comprising or, alternatively, consisting of Yl, Y2, Y3, Y4, Y5 and Y6.

In a preferred embodiment, the extraction solvent comprises at least a secondary aliphatic alcohol selected between isopropyl and isobutyl alcohol (Group C4).

In a preferred embodiment, the extraction solvent comprises at least a tertiary aliphatic alcohol.

Advantageously, the alcohol is t-butyl alcohol (Group C5).

Advantageously, the compound having at least an alcohol group (C1) can be selected from among the compounds having the formula (VI) [H—(O—CH₂—CH2—)ₙOH], where n can be comprised from 1 to 25, preferably from 2 to 20, even more preferably from 4 to 15 (Group C6).

All of the above-defined compounds (Groups C2-C6) belong to the group of compounds C1.

The following are some examples of (1) non-polar solvents, (2) aprotic polar solvents and (3) protic polar solvents, along with their dielectric constant value: (1) pentane, 1.84; hexane, 1.88; diethyl ether, 4.3; (2) ethyl acetate, 6.02; (3) formic acid, 58; n-butanol, 18; isopropanol, 18; n-propanol, 20; ethanol, 30; acetic acid, 6.2; water, 80.

In a preferred embodiment, the extraction solvent (A) comprises, or alternatively consists of, at least one compound belonging to group A1 in a mixture with water.

In another preferred embodiment, the extraction solvent (A) comprises, or alternatively consists of, at least one compound belonging to group B1 in a mixture with water.

In another preferred embodiment, the extraction solvent (A) comprises, or alternatively consists of, at least one compound belonging to group C1 in a mixture with water.

In another preferred embodiment, the extraction solvent (A) comprises, or alternatively consists of, at least one compound belonging to group B1 and at least one compound belonging to group C1 in a mixture with water.

The extraction method of the present invention envisages the use of an extraction liquid. The extraction liquid comprises, or alternatively consists of, an extraction solvent (A) and an extraction gas (Y), as defined above.

The extraction solvent can comprise polar and/or non polar solvent, of an aliphatic and/or aromatic nature (Group A1), in an amount of 1 to 60% by weight, relative to the total weight of the solvent, and water in an amount of 99 to 40% by weight, relative to the total weight of the solvent.

The extraction solvent can comprise at least one compound belonging to group B1 in an amount comprised from 1 to 60% by weight, relative to the total weight of the solvent, and water in an amount comprised from 99 to 40% by weight, relative to the total weight of the solvent.

The extraction solvent can comprise at least one compound belonging to group C1 in an amount comprised from 1 to 20% by weight, relative to the total weight of the solvent, and water in an amount comprised from 99 to 80% by weight, relative to the total weight of the solvent.

The extraction solvent can comprise at least one compound belonging to group B1 in an amount comprised from 1 to 60% by weight, relative to the total weight of the solvent, at least one compound belonging to group C1 in an amount comprised from 1 to 20% by weight, relative to the total weight of the solvent and water in an amount comprised from 98 to 20% by weight, relative to the total weight of the solvent.

The extraction solvent has a pH comprised from 1 to 7, preferably from 2 to 6, even more preferably from 3 to 4, depending on the type of solvent used.

The extraction liquid comprising the extraction solvent and the extraction gas has a pH value comprised from 1 to 7, preferably from 2 to 6, even more preferably from 3 to 4, depending on the type of solvent used and the quantity of extraction gas added.

The extraction solvent comprises, or alternatively consists of acetic acid in an amount comprised from 1 to 80% by weight, relative to the total weight of the solvent, and water in an amount comprised from 99 to 20% by weight, relative to the total weight of the solvent. The extraction gas is carbon dioxide, or argon, or nitrogen, or carbon dioxide and argon, or argon and nitrogen, or carbon dioxide and argon and nitrogen and is present in a concentration comprised from 0.1 to 10% or from 0.5 to 5% by volume, relative to 100 parts by volume of extraction solvent used. Advantageously, acetic acid is present in an amount of 20% or 40% or 80% by weight, and the carbon dioxide, or argon, or nitrogen, or carbon dioxide and argon, or argon and nitrogen, or carbon dioxide and argon and nitrogen in an amount equal to 2.5% or 3% by volume.

In a preferred embodiment, the extraction solvent comprises, or alternatively consists of, ethyl alcohol in an amount comprised from 1 to 20% by weight, relative to the total weight of the solvent, and water in an amount comprised from 99 to 80% by weight, relative to the total weight of the solvent. The extraction gas is carbon dioxide and is present in a concentration comprised from 0.5 to 5% by volume, relative to 100 parts by volume of extraction solvent used. Advantageously, the ethyl alcohol is present in an amount of 10% by weight and carbon dioxide 3% by volume.

In a preferred embodiment, the extraction solvent comprises, or alternatively consists of acetic acid in an amount comprised from 1 to 60% by weight, relative to the total weight of the solvent, and ethyl alcohol in an amount comprised from 1 to 20% by weight, relative to the total weight of the solvent, and water in an amount comprised from 98 to 20% by weight, relative to the total weight of the solvent.

The extraction gas is carbon dioxide, or argon, or nitrogen, or carbon dioxide and argon, or argon and nitrogen, or carbon dioxide and argon and nitrogen and is present in a concentration comprised from 0.1 to 10% or from 0.5 to 5% by volume, relative to 100 parts by volume of extraction solvent used.

Advantageously, the acetic acid is present in an amount of 20% or 40% or 80% by weight, the ethyl alcohol is present in an amount of 10% by weight and the carbon dioxide or argon 3% by volume, or carbon dioxide and argon together in an amount of 2.5% each.

In a preferred embodiment, the extraction solvent comprises, or alternatively consists of, acetic acid in an amount comprised from 1 to 60% by weight, relative to the total weight of the solvent, ethyl alcohol in an amount comprised from 1 to 10% by weight, relative to the total weight of the solvent, ethyl ethanoate in an amount comprised from 1 to 10% by weight, relative to the total weight of the solvent and water in an amount comprised from 97 to 20% by weight, relative to the total weight of the solvent. The extraction gas is carbon dioxide and is present in a concentration comprised from 0.5 to 5% by volume, relative to 100 parts by volume of extraction solvent used. Advantageously, the ethyl alcohol is present in an amount of 10% by weight and the carbon dioxide 3% by volume.

In a preferred embodiment, the extraction solvent comprises, or alternatively consists of, ethyl alcohol in an amount comprised from 1 to 10% by weight, relative to the total weight of the solvent, ethyl ethanoate in an amount comprised from 1 to 10% by weight, relative to the total weight of the solvent and water in an amount comprised from 98 to 80% by weight, relative to the total weight of the solvent. The extraction gas is carbon dioxide and is present in a concentration comprised from 0.5 to 5% by volume, relative to 100 parts by volume of extraction solvent used. Advantageously, the ethyl alcohol is present in an amount of 10% by weight and the carbon dioxide 3% by volume.

The acetic acid is preferably a 12% solution of acetic acid and the ethyl alcohol or ethanol is preferably 96% volume pure ethanol, known to those skilled in the art, having a maximum content of contaminants of approximately 0.058 mg/l. The water is double distilled water.

The extraction method of the present invention is conducted at an extraction temperature comprised from 20 to 90° C. Preferably, the extraction temperature is comprised from 25 to 65° C. Even more preferably, the extraction temperature is comprised from 40 to 60° C.

The extraction method of the present invention is conducted with an extraction time comprised from 1 to 8 hours. Preferably, the extraction time is comprised from 1.5 to 6 hours. Even more preferably, the extraction time is comprised from 3 to 5 hours.

In a preferred embodiment, the temperature at which the extraction method of the present invention is conducted is comprised from 25 to 65° C., preferably from 30 to 50° C., and the extraction time is comprised from 2 to 6 hours, preferably from 4 to 5 hours.

The extraction method of the present invention is conducted at an pressure comprised from 1 to 5 atmospheres, in the phase of equilibrium, preferably from 1.5 to 3 atmospheres.

Surprisingly, the extraction method of the present invention enables a large number of active molecules to be extracted at a high concentration from a vegetable substrate selected from among those listed below, according to the operating conditions used.

The vegetable substrate (extraction substrate) is selected from the group comprising, or consisting of, natural resins, fossil resins, seeds, barks, leaves, algae, essential oils, roots, vegetables and fruit, without any limitation, as will be demonstrated in the experimental part that follows.

In the context of the present invention, natural resin means a natural resin of vegetable origin or a vegetable resin or an organic resin. A vegetable resin is a mixture produced from a plant, of a liposoluble type, comprising volatile and non-volatile terpene compounds and/or phenolic compounds.

Preferably, the method of the present invention has valid application with the substrates listed below, which can be, for example, in the form of bark, leaves, seeds, roots or resin: myrrh, incense (name generically attributed to the oleoresins secreted by various shrubs, for example by Boswellia sacra), Dacryodes (for example, Dacryodes belemensis, buettneri, edulis, excelsa, occidentalis, olivifera, peruviana, pubescens), Dammar (obtained from plants of the family Dipterocarpaceae, mainly of the genus Shorea, Balanocarpus or Hopea), Benzoin (Styrax benzoin Dryander or Styrax benzoides Craib, of the family Styracaceae. Fragrant tree or shrub of the Polycarpels), Guaranà (Paullinia cupana), Griffonia (Griffonia simplicifolia, Griffonia salicifolia), mandarin, liquorice, mint (Aquilaria malaccensis), Senna (Cassia angustifolia), Ginger, Rhubarb, Gingseng, Vaccinium (Cranberry, Blueberry, Bilberry), Blackberry, Chrysanthemum (Tanacetum parthenium), Frankincense (Boswellia carterii), Willow (plants of the genus Salix, family Salicaceae), amber, propolis (European propolis, Brazilian propolis, Indian propolis), tea, Artemisia (genus of plants belonging to the family Asteraceae), Cinnamon (Cinnamomum zeylanicum, Cinnamomum aromaticum), Acacia (genus of plants of the family Mimosaceae) and Valerian (Valeriana Officinalis).

The extraction method of the present invention is capable of extracting the molecules present in said vegetable substrates, selected from the group comprising terpenes, flavonoids, anthocyans and catechins.

In the context of the present invention, Terpenes (or Isoprenoids) means molecules consisting of multiples of isoprene units and which can be linear, cyclic or both. It is common also to indicate the various terpenoids with the word terpene.

Based on the number of isoprene units contained (C5H8), the classification is the following: Hemiterpenes (1 unit, carbon number 5); Monoterpenes (2 units, carbon number 10); Sesquiterpenes (3 units, carbon number 15); Diterpenes (4 units, carbon number 20); Sesterpenes (5 units, carbon number 25); Triterpenes (6 units, carbon number 30) and Tetraterpenes (8 units, carbon number 40).

In the context of the present invention, Flavonoids (or Bioflavonoids) means polyphenolic compounds. In particular, the various subclasses are: Flavones, derived from 2-phenyl-chromen-4-one (2-phenyl-1,4-benzopyrone); Isoflavones, derived from 3-phenyl-chromen-4-one (3-phenyl-1,4-benzopyrone) and Neoflavones, derived from 4-phenylcoumarin (4-phenyl-1,2-benzopyrone).

In the context of the present invention, Anthocyans (or Anthocyanins) means a class of water-soluble vegetable compounds belonging to the family of the flavonoids. Anthocyanins derive from their respective aglicones (anthocyanidins), from which they differ in the addition of a glycoside group. In nature about twenty aglicones exist, whereas the number of derivatives is up to 15-20 times greater. The first ones, most frequent in nature, include, for example: delphinidin, petunidin, cyanidin, anthocyanin, malvidin, peonidin, tricetinidin, apigeninidin, pelargonidin and proanthocyanin, whose names derive from plants rich in them.

In the context of the present invention, Catechin means a vast family of polyphenolic compounds which comprises the flavan-3-ols (catechins and their epicatechin isomers), selected from among: epigallocatechin-3-gallate (EGCG), epigallocatechin (EGC), epicatechin-3-gallate (ECG), epicatechin (EC), gallocatechin and catechin.

The mixtures of the different enantiomers are: (+/−) catechin or DL-catechin and (+/−)-epicatechin or DL-epicatechin. The epigallocatechins, for example epigallocatechin gallate (EGCG) are also included.

The method of the present invention envisages a step in which the vegetable substrate and/or the natural resins and/or the essential oils (raw materials), in the form of powders or granules having a particle size comprised from 10 to 200 microns, preferably from 20 to 100, even more preferably from 40 to 80, are loaded into a container, for example a tank, equipped with inlet and outlet means, stirring means, for example rotating blades, and heating means, for example a heating mantle. In the event that the vegetable substrate and/or the natural resins (raw materials) are of a dimension or in a physical form that is not suitable for extraction, for example in the form of dry resins, the vegetable substrate and/or the natural resins are submitted to mechanical crushing and/or grinding so that they can be transformed into powder or granules with a particle size suitable for extraction purposes.

Subsequently, a step is performed in which, for example, the natural resins present in said container for extraction are placed in contact with the extraction liquid, which consists of an extraction solvent and extraction gas, as described above. The extraction of the active molecules takes place under the conditions of temperature, time, pressure and pH as described above.

The ratio by weight between the extraction solvent and the vegetable substrate and/or natural resins is in comprised from 1:1 to 30:1, preferably from 5:1 to 25:1, even more preferably from 10:1 to 15:1.

Preferably, the extraction solvent is added to the vegetable substrate and/or natural resins and, subsequently, the extraction gas is added to the extraction solvent, for example by blowing or bubbling the extraction gas into the extraction solvent to give the extraction liquid and, subsequently, the extraction liquid begins to circulate, for an extraction time as indicated above, within the vegetable substrate and/or natural resins, giving rise to the extraction.

During the extraction process, the extraction liquid is kept circulating inside the apparatus through the use of pumping means and filtered through the use of filter means, for example, using a multiple-section filter pack.

Subsequently, the extraction liquid containing the extraction gas, the extraction solvent and the active molecules extracted from said vegetable substrate and/or natural resins is collected in a collection tank. The method can be carried out in a continuous or discontinuous mode. The extract obtained can be in liquid form or in the form of a dense liquid with high viscosity.

The subject matter of the present invention relates to a liquid extract obtained with the extraction method described above.

The extract, for example in liquid form, comprising the above-described active molecules, has a pH value that will depend on the type of extraction liquid used. In general, the pH of the extract is comprised from 1 to 7, for example from 1.5 to 5.5.

If the extraction liquid consists of an extraction solvent which comprises one or more acidic substances, as described above, the liquid extract will have a pH lower than 7. In such a case, the extract will be neutralized using an basic substance selected between magnesium carbonate and sodium hydroxide in order to reach neutrality. After neutralization, if this has been necessary, the liquid extract will have a pH of around 7.

The liquid extract of the present invention can be subjected to a solvent evaporation procedure or a drying or lyophilization procedure to give an extract in solid form (or a very dense/viscous extract), preferably in powder, granular or lyophilized form. This extract in solid form is used to prepare a food composition, a supplement product, a nutraceutic composition or a pharmaceutical product for internal or external use, preferably for topical or oral administration.

1) Dietary supplement in tablets.

One tablet contains:
Noxamicina® 50 mg (Propolis flavonoids) hydroalcoholic solution of Propolis titrated to 2.58% bioflavonoids (equal to 1.3 mg per tablet), obtained with the extraction process in accordance with the present invention.
Antiagglomerants: silicon dioxide and magnesium stearate, magnesium oxide.
Flavouring: strawberry

2) Dietary supplement in tablets.

One tablet contains:
Noxamicina® 50 mg (Propolis flavonoids), obtained with the extraction process in accordance with the present invention.
Cranberry extract 90 mg, titrated to 80% proanthocyanidins, equal to 72 mg, obtained with the extraction process in accordance with the present invention.
Antiagglomerants: silicon dioxide and magnesium stearate, magnesium oxide.
Flavouring: strawberry

3) Dietary supplement in tablets

One tablet contains:
Griffonia 25 mg (Griffonia simplicifolia) semi dry extract (titrated to 99% 5-hydroxytryptophan), obtained with the extraction process in accordance with the present invention.
Melatonin 5 mg.
Antiagglomerants: silicon dioxide and magnesium stearate.

4) Dietary supplement in drops
Water and fructose.
Thickening agent: vegetable glycerin.
Valerian (Valeriana officinalis) root, dry extract (to 0.8% valerenic acids, maltodextrin), obtained with the extraction process in accordance with the present invention.
Melatonin 5 mg (every 40 drops).
Citric acid.
Potassium sorbate

The subject matter of the present invention further relates to an apparatus for carrying out the extraction method of the present invention (FIG. 1).

FIG. 1 shows an apparatus 1 which comprises a tank 01 for containing a given amount of extraction solvent. The solvent is maintained under stirring through the use of stirring means 02. The extraction solvent is made to flow from the tank 01 to the tank 05 using a pump 03 and connection means 04. The tank 05 is equipped with heating means 06 and stirring means 09.

The substrate to be extracted is introduced into the tank 11 and is maintained under stirring through the use of stirring means 12 to avoid clogging.

The container 05 is heated with the heating means 06. The pumps 10 and 20 are then started and the extraction solvent begins circulating from the tank 05 to the tank 11 through connection means containing the pump 10. The extraction solvent inside the tank 11 comes into contact with the extraction substrate contained therein, forming a suspension which is maintained under stirring through the use of the stirring means 12. At this point, the extraction gas contained in the tank 07 is made to flow in through the pipe 08.

The extraction gas is carbon dioxide CO₂ (P=1 Atm and T=23° C.) and is introduced in a predefined amount. The extraction gas is introduced into the extraction solvent contained in the tank 05 to give the extraction liquid using a volumetric flow filler 10 for the gas. Once all of the extraction gas has been introduced, the volumetric flow filler 10 is shut off and the apparatus is brought into thermal and pressure equilibrium. In practice, the air present in the pipes is removed (degassing) and one waits until the extraction temperature is uniform and constant. Moreover, the pressure value at which the extraction will be performed is set. The apparatus is sealed off hermetically. At this point the extraction process begins. The tank 15 is connected to the container 11 using connection means 13 containing the filter means 14. Furthermore, the tank 15 is connected to the tank 05 using connection means 22 containing the pump 20 and the filter means 21. The tank 15 is equipped with stirring means 16. Finally, the tank 15 is connected to the collection tank 19 using connection means 18 containing the pump 17. The extraction liquid is continuously filtered through the filters 14 and 21. The liquid contained in the tank 15 is transferred into the collection tank 19 by means of the pump 17. Filtration and low-pressure evaporation of the extract yield a concentrated gel. The filtering station 21 consists of a filter packet which comprises a mesh filter of a metallic type (REP) having a pore size of 200 to 400 microns, preferably 250 to 300 microns, and two to four polypropylene or polyethylene fabric filters with a 5 micron filtration capacity. For example, the filter 14 comprises only one or a number of metal filters. The characteristics and advantages of the extraction method of the present invention will become more apparent from the detailed description that follows, which is set forth through the illustration of some examples which are not intended in any way to limit the scope of the present invention. The numbers refer to those present in the drawing of the extraction apparatus (FIG. 1).

### EXAMPLE N.1—MYRRH

The extraction method of the present invention was used to extract the active molecules contained in myrrh. The extraction apparatus was loaded with 1500 ml of extraction solvent comprising: acetic acid (12% aqueous solution), 58% by weight , and distilled water, 42% by weight. The solvent:myrrh ratio was 15:1 by weight; 100 g of myrrh was finely ground (particle size comprised from 100-120 microns). Carbon dioxide was used in an amount of 30 ml (2% by volume, relative to the total volume of extraction solvent). A predefined amount of extraction solvent (1500 ml) is introduced into a tank 01 and is maintained under stirring at 50 rpm for 15 minutes, using the stirring means 02. The extraction solvent is made to flow from the tank 01 to the tank 05 using a pump 03 and connection means 04. The tank 05 is equipped with heating means 06 and stirring means 09. 100 g of myrrh is weighed and ground (particle size comprised from 40-100 microns), introduced into the tank 11 and maintained under stirring using the stirring means 12 to avoid clogging. The container 05 is heated to 45° C. (+/−0.5° C.) with the heating means 06. The pumps 10 and 20 are then started and the extraction solvent begins to circulate from the tank 05 to the tank 11 through the connection means containing the pump 10. The extraction solvent inside the tank 11 comes into contact with the ground myrrh contained inside it, forming a suspension, which is maintained under stirring using the stirring means 12. At this point, the extraction gas contained in the tank 07 is made to flow in through the pipe 08. The extraction gas is carbon dioxide CO₂ (P=1 Atm and T=23° C.) and is introduced in an amount equal to 2% by volume, relative to the volume of the extraction solvent, and then in an amount equal to 30 ml. The extraction gas is introduced into the extraction solvent contained in the tank 05 to give the extraction liquid using a volumetric flow filler 10 for the gas. Once all of the extraction gas has been introduced, the volumetric flow filler 10 is shut off and the apparatus is brought into thermal and pressure equilibrium. In practice, the air present in the pipes is removed (degassing) and one waits until the extraction temperature is uniform and constant. Moreover, the pressure value at which the extraction will be performed is set. The apparatus works under hermetic conditions. At this point the extraction process begins; it lasts 5 hours at a temperature of 45° C. The tank 15 is connected to the container 11 using connection means 13 containing the filter means 14. Moreover, the tank 15 is connected to the tank 05 using connection means 22 containing the pump 20 and the filter means 21. The tank 15 is equipped with stirring means 16. Finally, the tank 15 is connected to the collection tank 19 using connection means 18 containing the pump 17. The extraction liquid is continuously filtered through the filters 14 and 21. Once the 5 hours have elapsed the liquid contained in the tank 15 is transferred into the collection tank 19 by means of the pump 17. Filtration and low-pressure evaporation of the extract yield a concentrated gel. The extraction liquid (liquid extract) or concentrated gel is submitted to qualitative analysis conducted by GC/MS gas chromatography (mass gas chromatography mass) and quantitative analysis conducted by GC/FID (Gas chromatography flame ionic detection) using a Hewlett-Packard HP 6890 and Hewlett-Packard HPLC Agilent 1100 (table 1). As may be seen from Table 1, 51 active molecules were extracted from the myrrh. The extraction yield by weight was 105,000 ppm, relative to the total by weight of the extractable substances initially present in the 100 g of myrrh, which corresponds to 10.5% for 100 g of myrrh.

### EXAMPLE N.2

### Incense

The extraction method of the present invention was used to extract the active molecules contained in incense. Example 2 was conducted with the same operating procedures as described in example 1, with the sole differences described below. The extraction apparatus was loaded with 1500 ml of extraction solvent comprising: acetic acid (12% aqueous solution), 58% by weight; 99% ethanol, 20% by weight, and distilled water, 22% by weight. The solvent:incense ratio by weight was 15:1; 100 g of incense was finely ground (particle size comprised from 100-120 microns). Carbon dioxide was used in an amount equal to 30 ml (2% by volume, relative to the total volume of extraction solvent). As may be seen from Table 2, 46 active molecules were extracted from the incense. The extraction yield by weight was 125,000 ppm relative to the total by weight of the extractable substances initially present in the 100 g of incense, which corresponds to 12.5% for 100 g of incense.

### EXAMPLE N.3

### Tanacetum Parthenium

The extraction method of the present invention was used to extract the active molecules contained in Tanacetum Parthenium (abbreviated TP). Example 3 was conducted with the same operating procedures as described in example 1, with the sole differences described below. The extraction apparatus was loaded with 1500 ml of extraction solvent comprising: acetic acid (12% aqueous solution), 40% by weight, and distilled water, 60% by weight. The solvent:TP ratio by weight was 15:1; 100 g of TP was finely ground (particle size comprised from 100-120 microns). Carbon dioxide was used in an amount equal to 45 ml (3% by volume, relative to the total volume of extraction solvent). As may be seen from Table 3, 49 active molecules were extracted from the incense. The extraction yield by weight was 85,000 ppm relative to the total by weight of the extractable substances initially present in the 100 g of TP, which corresponds to 8.5% for 100 g of TP.

### EXAMPLE N.4

### Cranberry

The extraction method of the present invention was used to extract the active molecules contained in cranberry. Example 4 was conducted with the same operating procedures as described in example 1, with the sole differences described below. The extraction apparatus was loaded with 1500 ml of extraction solvent comprising: acetic acid (aqueous solution al 12%), 40% by weight, and distilled water, 60% by weight. The solvent:cranberry ratio by weight was 15:1; 100 g of cranberry was finely ground (particle size comprised from 80-100 microns). Carbon dioxide was used in an amount equal to 45 ml (3% by volume, relative to the total volume of extraction solvent). As may be seen from Table 4, 43 active molecules were extracted from the cranberry. The extraction yield by weight was 270,000 ppm relative to the total by weight of the extractable substances initially present in the 100 g of cranberry, which corresponds to 27% for 100 g of cranberry.

### EXAMPLE N.5

### Propolis

The extraction method of the present invention was used to extract the active molecules contained in propolis. Example 5 was conducted with the same operating procedures as described in example 1, with the sole differences described below. The extraction apparatus was loaded with 1500 ml of extraction solvent comprising: acetic acid (12% aqueous solution), 40% by weight, and distilled water, 60% by weight. The solvent:propolis ratio by weight was 15:1; 100 g of propolis was finely ground (particle size comprised from 80-120 microns).

Carbon dioxide was used in an amount equal to 45 ml (3% by volume, relative to the total volume of extraction solvent). As may be seen from Table 5, 44 active molecules were extracted from the propolis. The extraction yield by weight was 125,000 ppm relative to the total by weight of the extractable substances initially present in the 100 g of propolis, which corresponds to 12.5% relative to 100 g of propolis.

The Applicant carried out example 1 (myrrh) and example 2 (incense) as described above using the extraction method of the present invention with and without the use of carbon dioxide CO₂. The results obtained with the extraction method of the present invention in the presence of carbon dioxide show a significantly higher extraction yield, compared to the extraction method of the present invention without the use of gaseous carbon dioxide at 23° C. and 1 atmosphere of pressure, as shown by the values below.

| Substrate | Extraction method with CO₂ | Extraction method without CO₂ |
|---|---|---|
| Myrrh | 105000 ppm | 66000 ppm |
| Incense | 125000 ppm | 75000 ppm |

A comparison between the total number of active molecules extracted with the method described in patent EP 1641903 B1 and the total number of the active molecules extracted with the extraction method of the present invention is shown below.

| Method | Total molecules extracted |
|---|---|
| Method of EP1641903B1 Myrrh | 28 |
| Present invention Myrrh | 51 |
| Method of EP1641903B1 Incense | 32 |
| Present invention Incense | 46 |
| Method of EP1641903B1 Propolis | 17 |
| Present invention Propolis | 44 |

Furthermore, compared to the method described in patent EP 1641903 B1, the method of the present invention is capable of extracting molecules having a molecular weight of up to 4000 daltons, whereas the previous method was able to reach 650 daltons.

The extract obtained with the method of the present invention was submitted to light scattering analysis with a ruby laser beam having a wavelength comprised from 5500-7500 Å. It was observed that the laser light did not give rise to the Tyndall effect. This confirms that the extract “solution” is one where the extracted molecules are free and isolated. Consequently, these extracted molecules are very active biologically, since they exhibit better molecular kinetics and high diffusibility in a lipid medium or across cellular barriers.

Advantageously, the molecules extracted with the method of the present invention are extracted in free form (not in the form of molecular aggregates) and are free of polymeric components present in vegetable substrates such as, for example, in natural resins, such as the gummy components, starchy components, sugars and proteins.

### EXAMPLE N.6 CRANBERRY

### Comparative Test

The extraction method of the present invention was used to extract the active molecules contained in cranberry. Example 6 was conducted with the same operating procedures as described in example 4, with the sole differences described below.

Composition of the Solvents (S1-S4):
S1: Acetic acid 80%, water 20% and 5% carbon dioxide (AA+CO2).
S2: Acetic acid 80%, water 20% and 2.5% carbon dioxide and 2.5% Argon (AA+CO₂+Ar₂).
S3: Acetic acid 80%, water 20% and 5% Argon (AA+Ar₂).
S4: Ethanol 80%, water 20% and carbon dioxide 5% (EtOH+CO₂).

Extraction parameters: T 40° C., extraction time 60 minutes and continuous filtration.

### EXAMPLE N.7 TANACETUM PARTHENIUM

### Comparative Test

The extraction method of the present invention was used to extract the active molecules contained in Tanacetum parthenium. Example 7 was conducted with the same operating procedures as described in example 3, with the sole differences described below.

Composition of the solvents:
S1: Acetic acid 80%, water 20% and 5% carbon dioxide (AA+CO2).
S2: Acetic acid 80%, water 20% and 2.5% carbon dioxide and 2.5% Argon (AA+CO₂+Ar₂).
S3: Acetic acid 80%, water 20% and 5% Argon (AA+Ar₂).
S4: Ethanol 80%, water 20% and carbon dioxide 5% (EtOH+CO₂).

Extraction parameters: T 45° C., extraction time 60 minutes and continuous filtration.

### EXAMPLE N.8 INCENSE

### Comparative Test

The extraction method of the present invention was used to extract the active molecules contained in incense. Example 8 was conducted with the same operating procedures as described in example 2, with the sole differences described below.

Composition of the solvents:
S1: Acetic acid 80%, water 20% and 5% carbon dioxide (AA+CO2).
S2: Acetic acid 80%, water 20% and 2.5% carbon dioxide and 2.5% Argon (AA+CO₂+Ar₂).
S3: Acetic acid 80%, water 20% and 5% Argon (AA+Ar₂).
S4: Ethanol 80%, water 20% and carbon dioxide 5% (EtOH+CO₂).

Extraction parameters: T 70° C., extraction time 60 minutes and continuous filtration.

### EXAMPLE N.9 MYRRH

### Comparative Test

The extraction method of the present invention was used to extract the active molecules contained in incense. Example 9 was conducted with the same operating procedures as described in example 1, with the sole differences described below.

Composition of the solvents:
S1: Acetic acid 80%, water 20% and 5% carbon dioxide (AA+CO2).
S2: Acetic acid 80%, water 20% and 2.5% carbon dioxide and 2.5% Argon (AA+CO₂+Ar₂).
S3: Acetic acid 80%, water 20% and 5% Argon (AA+Ar₂).
S4: Ethanol 80%, water 20% and carbon dioxide 5% (EtOH+CO₂).

Extraction parameters: T 70° C., extraction time 60 minutes and continuous filtration.

### EXAMPLE N.10 PROPOLIS

### Comparative Test

The extraction method of the present invention was used to extract the active molecules contained in incense. Example 10 was conducted with the same operating procedures as described in example 5, with the sole differences described below.

Composition of the solvents:
S1: Acetic acid 80%, water 20% and 5% carbon dioxide (AA+CO2).
S2: Acetic acid 80%, water 20% and 2.5% carbon dioxide and 2.5% Argon (AA+CO₂+Ar₂).
S3: Acetic acid 80%, water 20% and 5% Argon (AA+Ar₂).
S4: Ethanol 80%, water 20% and carbon dioxide 5% (EtOH+CO₂).

Extraction parameters: T 40° C., extraction time 120 minutes and continuous filtration.

TABLE 1
MYRRH - COMPOUNDS EXTRACTED - QUANTITATIVE

| Total quantity of compounds extracted: 105,000 ppm | | | | | |
|---|---|---|---|---|---|
| (concentration present in the solvent as solute) | | | Number of compounds present 51 | | |
| Germacrone | 900 | ppm | Germacrone | compound | 1 |
| Furanodiene | 1150 | ppm | Furanodiene | compound | 2 |
| 2-methoxyfuranodiene | 2500 | ppm | 2-metossifuranodiene | compound | 3 |
| 4,5-dihydrofuranodiene-6-one | 550 | ppm | 4,5-diidrossifuranodiene | compound | 4 |
| Beta-selinene | 300 | ppm | beta selinene | compound | 5 |
| Lindestrene | 2000 | ppm | lindestrene | compound | 6 |
| Furanoeudesma-1,3-diene | 15750 | ppm | furanoeudesma-1,3-diene | compound | 7 |
| Beta-elemene | 8400 | ppm | beta elemene | compound | 8 |
| Gamma-elemene | 2370 | ppm | gamma elemene | compound | 9 |
| Delta-elemene | 550 | ppm | delta elemene | compound | 10 |
| Elemon | 210 | ppm | elemon | compound | 11 |
| Isofuranogermacrene | 3385 | ppm | isofuranogermacrene | compound | 12 |
| Curzerenone | 1500 | ppm | curzerenone | compound | 13 |
| Alpha-cubebene | 1500 | ppm | alfa cubebene | compound | 14 |
| Beta-bourbonene | 450 | ppm | beta bourbonene | compound | 15 |
| Alpha-pinene | 210 | ppm | alfa pinene | compound | 16 |
| Myrcene | 150 | ppm | mircene | compound | 17 |
| Beta-ylangene | 150 | ppm | beta-ylangene | compound | 18 |
| Alpha-gurjunene | 500 | ppm | alfa-gurjunene | compound | 19 |
| Alloarmatoandrene | 2500 | ppm | alloaromatoandrene | compound | 20 |
| Bicyclogermacrene | 5000 | ppm | biciclogermacrene | compound | 21 |
| Alpha-guaiene | 2500 | ppm | alfa-guaiene | compound | 22 |
| Gamma-cadinene | 1500 | ppm | gamma cadinene | compound | 23 |
| Delta-cadinene | 800 | ppm | delta cadinene | compound | 24 |
| T-cadinol | 1700 | ppm | T-cadinolo | compound | 25 |
| Lindestrene | 200 | ppm | lindestrene | compound | 26 |
| Beta-sequiphellandrene | 150 | ppm | beta sequifellandrene | compound | 27 |
| 2-O-methyl.8,12-epoxygermacrane-1(10),4,7,11-tetraene isomer | 500 | ppm | unvaried | compound | 28 |
| O-acetyl-8,12-epoxygermacrane-1(10)4,7,11-tetraene isomer | 3900 | ppm | unvaried | compound | 29 |
| Bicyclogermacrene | 1800 | ppm | bicliclogermacrene | compound | 30 |
| Myrrhone | 300 | ppm | mirrone | compound | 31 |
| epicurzerenone | 2000 | ppm | epicurzerenone | compound | 32 |
| Eusesm-4(15)-ene-1Beta-6-alpha-diol | 850 | ppm | unvaried | compound | 33 |
| 4-O-methyl-glucuronic acid | 1500 | ppm | acido 4-O,metil-glucuronico | compound | 34 |
| Quertecin | 2500 | ppm | quercetina | compound | 35 |
| Quercectin-3-O-beta-D-galactoside | 200 | ppm | unvaried | compound | 36 |
| Guggulsterol | 4500 | ppm | guggulsterolo | compound | 37 |
| Quercetin-3-O-alpha-L-arabinose | 1500 | ppm | unvaried | compound | 38 |
| Ellagic acid | 1200 | ppm | acido ellagico | compound | 39 |
| Pelagornidin | 1300 | ppm | pelagordina | compound | 40 |
| Linoleic acid | 600 | ppm | acido linoleico | compound | 41 |
| Alpha mirrholic acid | 5700 | ppm | acido alfa mirrolico | compound | 42 |
| Beta mirrhoic acid | 5300 | ppm | acido beta mirrolico | compound | 43 |
| Gamma mirrholic acid | 4600 | ppm | acido gamma mirrolico | compound | 44 |
| Guggulsterone | 7800 | ppm | guggulsterone | compound | 45 |
| Bornyl acetate | 120 | ppm | bornil acetato | compound | 46 |
| d-limonene | 200 | ppm | d-limonene | compound | 47 |
| Linalool | 80 | ppm | linalolo | compound | 48 |
| methylcalvicol | 150 | ppm | metilclavicolo | compound | 49 |
| Alpha-terpineol | 250 | ppm | alfa terpineolo | compound | 50 |
| Dammarane triterpene | 1275 | ppm | unvaried | compound | 51 |
| | | | | Total compounds | 51 |

TABLE 2
INCENSE - COMPOUNDS EXTRACTED - QUANTITIVE

| Total quantity of compounds extracted: 125,000 ppm | | | | | |
|---|---|---|---|---|---|
| (concentration present in the solvent as solute) | | | Number of compounds present 46 | | |
| Beta pinene | 300 | ppm | beta pinene | compound | 1 |
| Alpha pinene | 3000 | ppm | alfa pinene | compound | 2 |
| Isoterpinolene | 50 | ppm | isoterpinolene | compound | 3 |
| Alpha-phellandrene | 40 | ppm | alfa fellandrene | compound | 4 |
| Beta-phellandrene | 40 | ppm | beta fellandrene | compound | 5 |
| d-limonene | 9500 | ppm | d-limonene | compound | 6 |
| Cis-ocimene | 500 | ppm | cis-ocimene | compound | 7 |
| Beta-citronellol | 300 | ppm | beta-citronellol | compound | 8 |
| Cis-carveol | 250 | ppm | cis-carveolo | compound | 9 |
| Trans-terpin | 600 | ppm | trans-terpin | compound | 10 |
| Carvone | 300 | ppm | carvone | compound | 11 |
| Piperitone | 40 | ppm | piperitone | compound | 12 |
| Alpha copaene | 35 | ppm | alfa copaene | compound | 13 |
| Delta selinene | 300 | ppm | celta selinene | compound | 14 |
| Viridifloor | 80 | ppm | viridifloor | compound | 15 |
| Maaliane | 75 | ppm | maaliane | compound | 16 |
| Alpha-muruulol | 40 | ppm | alfa murulolo | compound | 17 |
| Beta bisabolene | 380 | ppm | beta bisabolene | compound | 18 |
| Cis-calamandrene | 40 | ppm | cis-calamandrene | compound | 19 |
| Spathulenol | 200 | ppm | spatunelolo | compound | 20 |
| Cis-nerolidol | 300 | ppm | cis-nerolidolo | compound | 21 |
| Isocembrene | 150 | ppm | isocembrene | compound | 22 |
| Duva-4,18.13-trien-1,5 alpha-diol | 80 | ppm | unvaried | compound | 23 |
| Thumbergol | 5000 | ppm | unvaried | compound | 24 |
| Duva-3,9,13-trien-1,5alpha, diol-1-acetate | 26000 | ppm | unvaried | compound | 25 |
| Isophillociadene | 200 | ppm | unvaried | compound | 26 |
| Octyl acetate | 16000 | ppm | octil acetato | compound | 27 |
| Benzyl benzoate | 280 | ppm | benzil benzoato | compound | 28 |
| Cembrene | 200 | ppm | cembrene | compound | 29 |
| n-octanol | 80 | ppm | n-octanolo | compound | 30 |
| neryl acetate | 600 | ppm | neril acetato | compound | 31 |
| cis-retinal | 200 | ppm | cis-retinale | compound | 32 |
| farnesyl acetate | 150 | ppm | fansesil acetato | compound | 33 |
| neryl acetate | 100 | ppm | neril acetato | compound | 34 |
| verbenone | 1550 | ppm | verbenone | compound | 35 |
| ursolic acid | 2500 | ppm | acido ursolico | compound | 36 |
| Alpha amyrin | 1500 | ppm | alfa amirina | compound | 37 |
| Epilupeol | 1200 | ppm | epilupeolo | compound | 38 |
| Mansubinol | 800 | ppm | mansubiolo | compound | 39 |
| Phytol | 1300 | ppm | fitolo | compound | 40 |
| Aromadendrene | 1200 | ppm | aromadendrene | compound | 41 |
| Beta boswellic acid (Beta-BA) | 8000 | ppm | acido beta boswellico | compound | 42 |
| 3-O-acetyl-beta-boswellic acid (A-beta-BA) | 8000 | ppm | unvaried | compound | 43 |
| 11-keto-beta bosewllic acid (KBA) | 6500 | ppm | unvaried | compound | 44 |
| 3-O-acetyl.11.k35o bosewllic acid (AKBA) | 24.300 | ppm | unvaried | compound | 45 |
| Alpha boswellic acid | 2000 | ppm | acido alfa bosewllico | compound | 46 |
| | | | | Total compounds | 46 |

TABLE 3
TANICETUM PATHENIUM - COMPOUNDS EXTRACTED - QUANTITATIVE

| Total quantity of compounds extracted: 85,000 ppm | | | | |
|---|---|---|---|---|
| (concentration present in the solvent as solute) | | | Number of compounds present 49 | |
| Cis-2-octene | 35 | ppm | cis-2-octene | 1 |
| Butyl acetate | 750 | ppm | butil acetato | 2 |
| Tricyclene | 30 | ppm | triciclene | 3 |
| Alpha pinene | 25 | ppm | alfa pinene | 4 |
| Camphene | 900 | ppm | canfene | 5 |
| Sabinene | 7950 | ppm | sabinene | 6 |
| Beta pinene | 180 | ppm | beta pinene | 7 |
| Beta myrcene | 700 | ppm | beta mircene | 8 |
| p-cymene | 250 | ppm | p-cimene | 9 |
| limonene | 100 | ppm | limonene | 10 |
| Beta-phellandrene | 20 | ppm | beta fellandrene | 11 |
| 1,8 cineole | 1450 | ppm | 1,8 cineolo | 12 |
| Terpinolene | 20 | ppm | terinolene | 13 |
| Terpin-1-ol | 600 | ppm | terpin-2-ol | 14 |
| Camphor | 25000 | ppm | casnfora | 15 |
| E-chrstanthemyl acetate | 23250 | ppm | E-cristantemil acetato | 16 |
| Pinocarvone | 20 | ppm | pinocarvone | 17 |
| Borneol | 90 | ppm | borneolo | 18 |
| Alpha terpineol | 300 | ppm | alfa terpineolo | 19 |
| Linalool acetate | 480 | ppm | linalolo acetato | 20 |
| Bornyl angelate | 3750 | ppm | bornil angelate | 21 |
| Pinocarvone | 20 | ppm | pinocarvone | 22 |
| Borneol | 200 | ppm | borneolo | 23 |
| Citronellal hydrate | 20 | ppm | citronellal idrato | 24 |
| Thymol | 2450 | ppm | timolo | 25 |
| E-pinocarvyl acetate | 700 | ppm | E-pinocarvil acetato | 26 |
| Carvacrol | 20 | ppm | carvacrolo | 27 |
| Alpha-copaene | 20 | ppm | alfa copaene | 28 |
| E-caryophillene | 230 | ppm | E-cariofillene | 29 |
| Alpha-humulene | 200 | ppm | alfa humulene | 30 |
| Germacrene-D | 180 | ppm | germacrene-D | 31 |
| Ar-curcumene | 20 | ppm | Ar-curcumene | 32 |
| Isobornyl-2.methyl butyrate | 20 | ppm | unvaried | 33 |
| Sigma-cadinene | 20 | ppm | sigma cadinene | 34 |
| Z-Christanthenyl acetate | 1500 | ppm | Z-cristantemil acetato | 35 |
| Bornyl acetate | 2600 | ppm | bornil acetato | 36 |
| Viridifloor | 20 | ppm | viridifloor | 37 |
| Globulol | 4950 | ppm | globulol | 38 |
| E,E farnesol | 200 | ppm | E,E farnesolo | 39 |
| Santin | 150 | ppm | santin | 40 |
| Apigenin | 1250 | ppm | apigenina | 41 |
| Luteolin | 1250 | ppm | luteolina | 42 |
| Quercetin | 1350 | ppm | quercetina | 43 |
| Beta-amyrin | 500 | ppm | beta amirina | 44 |
| Beta-sitosterol | 300 | ppm | beta sitosterolo | 45 |
| Parthenolide | 1500 | ppm | prtenolide | 46 |
| Epoxysantamarin | 150 | ppm | epossisantamarin | 47 |
| 3-beta-hydroxyparthenolide | 300 | ppm | 3-beta idrossipartenolide | 48 |
| Lutein | 50 | ppm | luteina | 49 |
| | | | | 49 |

TABLE 4
CRANBERRY - COMPOUNDS EXTRACTED - QUANTITATIVE

| Total quantity of compounds extracted: 270000 ppm | | | | | |
|---|---|---|---|---|---|
| (concentration present in the solvent as solute) | | | Number of compounds present 43 | | |
| Ascorbic acid (Vitamina C) | 32000 | ppm | acido ascorbico | compound 1 | 1 |
| Tridecanoic acid | 270 | ppm | acido tridecanoico | compound 1 | 2 |
| Heptadecanoic acid | 54 | ppm | acido eptadecanoico | compound | 3 |
| Eicosanoic acid | 270 | ppm | acido eicosanoico | compound 1 | 4 |
| Triconanoic acid | 54 | ppm | acido tricosanoico | compound 1 | 5 |
| Tetracosanoic acid | 110 | ppm | acido tetracosanoico | compound 1 | 6 |
| Linoileic acid | 35000 | ppm | acido linoleico | compound 1 | 7 |
| Oleic acid | 147000 | ppm | acido oleico | compound 1 | 8 |
| Ursolic acid | 3000 | ppm | acido ursolico | compound 1 | 9 |
| Palmitic acid | 30000 | ppm | acido palmitico | compound 1 | 10 |
| Cyclopentanoic acid | 150 | ppm | acido ciclopentanoico | compound 1 | 11 |
| 4-Hexadecen-6-yne | 200 | ppm | unvaried | compound 1 | 12 |
| Dodecosanoic acid | 270 | ppm | acido dodecosanoico | compound 1 | 13 |
| Squalene | 130 | ppm | squalene | compound 1 | 14 |
| Tocopherol beta | 30 | ppm | beta tocoferolo | compound 1 | 15 |
| Amyrin | 20 | ppm | amirina | compound 1 | 16 |
| Beta sistosterol | 3000 | ppm | beta sistosterolo | compound 1 | 17 |
| Sigmasterol | 300 | ppm | sigasterolo | compound 1 | 18 |
| Selenium | 10 | ppm | selenio | compound 1 | 19 |
| Vitamin A | 200 | ppm | vitamina A | compound 1 | 20 |
| Beta carotene | 2500 | ppm | beta carotene | compound 1 | 21 |
| Lutein | 150 | ppm | luteina | compound 1 | 22 |
| Ellagic acid | 300 | ppm | acido ellagico | compound 1 | 23 |
| Quercetin | 200 | ppm | quercetina | compound 1 | 24 |
| Myricetin | 150 | ppm | miricetina | compound 1 | 25 |
| Reseveratrol | 300 | ppm | reseveratrolo | compound 1 | 26 |
| Ferulic acid | 300 | ppm | acido ferulico | compound 1 | 27 |
| Cyanidin | 500 | ppm | cianidina | compound 1 | 28 |
| Peonidin | 100 | ppm | peonidina | compound 1 | 29 |
| Vanillic acid | 80 | ppm | acido vanillico | compound 1 | 30 |
| Caffeic acid | 150 | ppm | acido caffeico | compound 1 | 31 |
| Epilupeol | 150 | ppm | epilupeolo | compound 1 | 32 |
| 2,3-dihydroxy benzoic acid | 200 | ppm | acido 2,3-diidrossibenzoico | compound 1 | 33 |
| 2,4-dihydroxy benzoic acid | 370 | ppm | acido 2,4-diidrossibenzoico | compound 1 | 34 |
| Hyperoside | 100 | ppm | iperoside | compound 1 | 35 |
| Chlorgenic acid | 250 | ppm | acido clorogenico | compound 1 | 36 |
| Cyanidin-3-galactoside | 4000 | ppm | unvaried | compound 1 | 37 |
| Cyanidin-3-glucoside | 6000 | ppm | unvaried | compound 1 | 38 |
| Peonidin-3-glucoside | 1500 | ppm | unvaried | compound 1 | 39 |
| Tocopherol gamma | 24 | ppm | gamma tocoferolo | compound 1 | 40 |
| Dodecosanoic acid | 15 | ppm | acido dodecosanoico | compound 1 | 41 |
| Campesterol | 508 | ppm | campesterolo | compound 1 | 42 |
| Hexadecanoic acid | 95 | ppm | acido esadecanoico | compound 1 | 43 |
| | | | | TOTAL | 43 |
| | | | | COMPOUNDS | |

TABLE 5
PROPOLIS - COMPOUNDS EXTRACTED - QUANTITIVE

| Total quantity of compounds extracted: 125,000 ppm | | | | | |
|---|---|---|---|---|---|
| (final concentration present in the solvent as solute) | | | Number of compounds present 44 | | |
| Benzoic acid | 2000 | ppm | acido benzoico | compound 2 | 1 |
| Dihydrocinnamic acid | 500 | ppm | acido di-idrocinnamico | compound 2 | 2 |
| Z-cinnamic acid | 20000 | ppm | acido Z-cinnamico | compound | 3 |
| 3-phenyl-e-hydroxypropanoic acid | 5000 | ppm | acido-3.fenil-3-idropropanoico | compound | 4 |
| Methoxyphenylpropanoic acid | 750 | ppm | acido metossifenilpropanoico | compound | 5 |
| 4-hydroxybenzoic acid | 750 | ppm | acido 4-idrobenzoico | compound | 6 |
| Z-p-coumaric acid | 21 | ppm | acido z-p-cumarico | compound | 7 |
| E-p-coumaric acid | 4300 | ppm | acido E-p-cumarico | compound 2 | 8 |
| Ferulic acid | 1200 | ppm | acido ferulico | compound | 9 |
| Caffeic acid | 20 | ppm | acido caffeico | compound | 10 |
| Benzyl Alcohol | 125 | ppm | alcool benzilico | compound | 11 |
| Hidroquinone | 750 | ppm | Idrochinone | compound | 12 |
| 4-hydroxybenzilaldehyd | 350 | ppm | 4-idrossibenzaldeide | compound | 13 |
| cinnamic alcohol | 500 | ppm | alcool cinnamico | compound | 14 |
| Hydroxy acetophenone | 30000 | ppm | idrossi acetofenone | compound | 15 |
| Oleic acid | 20 | ppm | acido oleico | compound | 16 |
| Stearic acid | 370 | ppm | acido stearico | compound | 17 |
| Palmitic acid | 20 | ppm | acido palmitico | compound | 18 |
| Benzyl Benzoate | 4500 | ppm | benzil benzoato | compound | 19 |
| Benzyl methoxybenzoate | 8500 | ppm | benzil metossibenzoato | compound | 20 |
| Benzyl-Z-coumarate | 250 | ppm | benzil-Z-cumarato | compound | 21 |
| Benzyl ferulate | 1500 | ppm | benzil ferulato | compound | 22 |
| Benzyl caffeate | 1500 | ppm | benzil caffeato | compound | 23 |
| Phenethyl caffeate | 1800 | ppm | feniletil caffeato | compound | 24 |
| Cynnamil caffeate | 1200 | ppm | cinnamil caffeato | compound | 25 |
| Pinostrobin chalcone | 350 | ppm | pinostrobin calcone | compound | 26 |
| Pinocembrin | 2000 | ppm | pinocembrina | compound | 27 |
| Pinobanksin | 250 | ppm | pinobanksina | compound | 28 |
| Sakuratenin | 20 | ppm | sakuranetina | compound | 29 |
| Galangin | 1800 | ppm | galangina | compound | 30 |
| Quercetin | 1500 | ppm | quercetina | compound | 31 |
| Pinobaksin-3-O-acetate | 600 | ppm | pinobaksina-3-O-acetato | compound | 32 |
| Glicerol | 150 | ppm | glicerolo | compound | 33 |
| Kaempferol | 8000 | ppm | kaempferolo | compound | 34 |
| Farnesol | 4500 | ppm | farbesolo | compound | 35 |
| Apigenin | 1500 | ppm | apigenina | compound | 36 |
| Alpinone | 2000 | ppm | alpinone | compound | 37 |
| Morin | 1500 | ppm | morina | compound | 38 |
| 2′,6′-Dihydroxy-4′-methoxydihydrochalcone | | ppm | unvaried | compound | 39 |
| 2′,4′-6′-Trihydroxydihydrochalcone | 4000 | ppm | unvaried | compound | 40 |
| Isokuranetin | 2000 | ppm | isokuranetina | compound | 41 |
| Butyl caffeate | 1500 | ppm | butil caffeato | compound | 42 |
| Mististic acid | 1500 | ppm | acido miristico | compound | 43 |
| Beta-phenyl-ethyl-caffeate | 1500 | ppm | beta-fenil-etil-caffeato | compound | 44 |
| | | | | TOTAL | 44 |
| | | | | COMPOUNDS | |

TABLE 6

| | S1 | S2 | S3 | S4 |
|---|---|---|---|---|
| COMPOUND | ppm | ppm | ppm | ppm |
| Ascorbic acid/acido ascorbico | 32000 | 49000 | 68000 | 18000 |
| Tridecanoic acid/acido tridecanoico | 270 | 500 | 1200 | 250 |
| Heptadedanoic acid/acido eptanoico | 54 | 200 | 400 | 50 |
| Eicosanoic acid/acido eiconanoico | 270 | 500 | 600 | 200 |
| Tricosanoic acid/acido tricosanoico | 54 | 125 | 200 | 50 |
| Tetracosanoic acid/acido | 110 | 250 | 500 | 80 |
| tetracosanoico | | | | |
| Licoleic acid/acido linoleico | 35000 | 42000 | 62000 | 28000 |
| Oleic acid/acido oleico | 147000 | 185000 | 270000 | 95000 |
| Ursolic acid/acido ursolico | 3000 | 6000 | 9000 | 2000 |
| Palmitic acid/acido palimitico | 30000 | 42000 | 56000 | 25000 |
| Cyclopentamoic acid/acido | 150 | 250 | 400 | 20 |
| ciclopentanoic | | | | |
| 4-Hexadecen-6-yne/4-esadecen-6-ine | 200 | 400 | 500 | 50 |
| Dodecosanoicacid/acido | 270 | 300 | 600 | 100 |
| dodecosanoico | | | | |
| Squalene/squalene | 130 | 300 | 600 | 20 |
| Tocopherol beta/beta tocoferolo | 30 | 150 | 200 | 30 |
| Amyrin/amirina | 20 | 100 | 200 | 15 |
| Beta-sistosterol/beta-sistosterolo | 3000 | 5000 | 9000 | 2500 |
| Selenium/selenio | 10 | 40 | 150 | 10 |
| Vitamin A/vitamina A | 200 | 600 | 1250 | 50 |
| Beta-carotene/beta-carotene | 2500 | 3500 | 5000 | 1200 |
| Lutein/luteina | 150 | 300 | 1200 | 60 |
| Ellargic acid/acido ellargico | 300 | 600 | 1000 | 100 |
| Quercetin/quercetina | 200 | 400 | 800 | 80 |
| Myricetin/miricetina | 150 | 300 | 600 | 95 |
| Resveratrol/resveratrolo | 300 | 900 | 1500 | 120 |
| Ferulic acid/acido ferulico | 300 | 900 | 1500 | 150 |
| Cyanidin/cianidina | 500 | 1000 | 2100 | 200 |
| Peonidin/peonidina | 100 | 300 | 350 | 50 |
| Vanillic acid/acido vanillico | 80 | 100 | 200 | 35 |
| Caffeic acid/acido caffeico | 150 | 300 | 650 | 100 |
| Epilupeol/epilupeolo | 150 | 300 | 650 | 100 |
| 2,3-dihydroxybenzoic acid/ | 200 | 400 | 800 | 90 |
| Acido 2,3-diidrossibenzoico | | | | |
| 2,4-dihydroxybenzoic acid/ | 370 | 750 | 1500 | 150 |
| Acido 2,4-diidrossibenzoico | | | | |
| Hyperoside/Iperoside | 100 | 200 | 400 | 50 |
| Chlorogenic acid/Acido clorogenico | 250 | 600 | 1800 | 120 |
| Cyanidin-3.galactoside/unvaried | 4000 | 5000 | 10000 | 3000 |
| Cyanidin-3-glucoside/unvaried | 6000 | 7500 | 15000 | 3200 |
| Peonidin-3.glucoside/unvaried | 1500 | 3000 | 6000 | 800 |
| Gamma-tocopherol/gamma tocoferolo | 24 | 50 | 100 | 20 |
| Dodecosanoic acid/acido | 15 | 25 | 70 | 15 |
| dodecosanoico | | | | |
| Campesterol/campesterolo | 508 | 1000 | 2000 | 200 |
| Hexadecanoic acid/acido | 95 | 150 | 320 | 80 |
| esadecanoico | | | | |
| TOTAL QUANTITIES EXTRACTED | 270000 | 360490 | 538720 | 181320 |

TABLE 7

| | S1 | S2 | S3 | S4 |
|---|---|---|---|---|
| COMPOUND | ppm | ppm | ppm | ppm |
| Cis-2-octene/unvaried | 35 | 80 | 120 | 30 |
| Butyl acetate/butilacetato | 750 | 1200 | 2100 | 680 |
| Tricyclene/triciclene | 30 | 60 | 100 | 25 |
| Alpha-pinene/alfa pinene | 25 | 50 | 100 | 20 |
| Camphene/canfene | 900 | 1100 | 1800 | 800 |
| Sabinene/unvaried | 7950 | 9500 | 12000 | 6800 |
| Beta pinene/unvaried | 180 | 300 | 480 | 150 |
| Beta myrcene/unvaried | 700 | 1000 | 1800 | 600 |
| p-cymene/unvaried | 250 | 450 | 800 | 200 |
| Limonene/unvaried | 100 | 180 | 400 | 80 |
| Beta-phellandrene/unvaried | 20 | 35 | 80 | 20 |
| 1.8 cineole/1.8 cineolo | 1450 | 2250 | 3000 | 1200 |
| Terpinolene/unvaried | 20 | 35 | 80 | 15 |
| Terpin-1-o/unvaried | 600 | 900 | 1200 | 500 |
| Camphor/canfora | 25000 | 28000 | 32000 | 20000 |
| E-chrysantemyl acetate/ | 23250 | 30000 | 420000 | 19500 |
| E-crisantemil acetato | | | | |
| Pinocarvone/unvaried | 20 | 50 | 110 | 20 |
| Borneol/bomeolo | 90 | 120 | 270 | 80 |
| Alpha terpineol/alfa terpineolo | 300 | 500 | 900 | 250 |
| Linalol acetate/linalolo acetato | 480 | 750 | 1200 | 400 |
| Bornyl angelate/bornil angelato | 3750 | 6500 | 9000 | 3200 |
| Pinocarvone/unvaried | 20 | 30 | 60 | 20 |
| Borneol/bomeolo | 200 | 400 | 600 | 180 |
| Citronellal hydrate/citronellal idrato | 20 | 45 | 80 | 15 |
| Thymol/timolo | 2450 | 4500 | 6000 | 1900 |
| E-pinocarvone acetate/ | 700 | 1200 | 2400 | 650 |
| E-pinocarvone acetato | | | | |
| Carvacrol/carvacrolo | 20 | 40 | 160 | 20 |
| Alpha-copaene/alfa-copaene | 20 | 40 | 120 | 20 |
| E-caryophillene/E-cariofillene | 230 | 320 | 600 | 190 |
| Alpha-humulene/alfa-humulene | 200 | 400 | 620 | 150 |
| Germacrene-D/unvaried | 180 | 250 | 400 | 150 |
| Ar-Curcumene/unvaried | 20 | 60 | 120 | 20 |
| Isobornyl-2-methyl butyrate/ | 20 | 60 | 130 | 15 |
| isobornil-2-metil-butirrato | | | | |
| Sigma-cadinene/unvaried | 20 | 40 | 120 | 20 |
| Z-christantemil acetate/ | 1500 | 1950 | 2500 | 1300 |
| Z-crisantemil acetato | | | | |
| bornyl acetate/bornil acetato | 2600 | 3200 | 4000 | 2000 |
| Viridifloor/unvaried | 20 | 40 | 85 | 15 |
| Globulol/glubulolo | 4950 | 7000 | 11000 | 3800 |
| E,E farnesol/E,E farnesolo | 200 | 400 | 600 | 180 |
| Santin/unvaried | 150 | 320 | 450 | 100 |
| Apigenin/apigenina | 1250 | 2500 | 3500 | 800 |
| Luteolin/luteolina | 1250 | 2500 | 3500 | 900 |
| Quercetin/quercetina | 1250 | 2670 | 3850 | 1000 |
| beta-amyrin/beta amirina | 500 | 900 | 1200 | 350 |
| beta-sistosterol/beta-sistosterolo | 300 | 600 | 900 | 200 |
| Epoxysantamarin/epossisantamarin | 150 | 280 | 350 | 100 |
| 3-beta-hydroparthenolide/ | 150 | 250 | 350 | 100 |
| 3-beta-idropartenolide | | | | |
| Lutein/luteina | 50 | 100 | 300 | 20 |
| TOTAL QUANTITIES EXTRACTED: | 85000 | 115355 | 156285 | 68935 |

TABLE 8

| | S1 | S2 | S3 | S4 |
|---|---|---|---|---|
| COMPOUND | ppm | ppm | ppm | ppm |
| Beta-pinene/unvaried | 300 | 1500 | 2500 | 250 |
| alpha-pinene/unvaried | 3000 | 4000 | 8000 | 2500 |
| Isoterpinolene/unvaried | 40 | 450 | 800 | 40 |
| alpha-phellandrene/alfa fellandrene | 40 | 450 | 800 | 35 |
| beta-phellandrene/beta fellandrene | 40 | 280 | 800 | 35 |
| d-limonene/unvaried | 9500 | 12500 | 15000 | 7500 |
| cis-ocimene/unvaried | 500 | 2000 | 6000 | 450 |
| beta-citronellol/beta-citronellolo | 250 | 850 | 5000 | 250 |
| cis-carveol/cis carveolo | 600 | 1200 | 1900 | 500 |
| trans-terpin/unvaried | 600 | 850 | 2000 | 450 |
| Carvone/unvaried | 40 | 450 | 2500 | 40 |
| Piperitone/unvaried | 40 | 400 | 2500 | 40 |
| alpha-copaene/unvaried | 35 | 380 | 2400 | 30 |
| delta selinene/unvaried | 80 | 250 | 550 | 60 |
| Viridifloor/unvaried | 80 | 250 | 550 | 60 |
| Maaliane/unvaried | 75 | 205 | 550 | 55 |
| alpha-muruulol/alfa muruulolo | 40 | 140 | 500 | 35 |
| beta-bisabolene/unvaried | 380 | 450 | 800 | 250 |
| cis-calamandrene/unvaried | 40 | 380 | 600 | 40 |
| Spathulenol/spatulenolo | 200 | 600 | 1200 | 200 |
| cis-nerolidol/cis-nerolidolo | 300 | 950 | 2150 | 250 |
| Isocembrene/unvaried | 150 | 450 | 900 | 100 |
| duva-4,8,13-trien-1,alpha-diol | 80 | 250 | 600 | 70 |
| 250/unvaried | | | | |
| Thunbergol/tunbergolo | 5000 | 7800 | 9500 | 4000 |
| duva-3,9,13-trien-1,alpha,diol/ | 26000 | 37500 | 45800 | 20000 |
| Acetate/unvaried | | | | |
| Isophillociadene/isofillociadene | 200 | 800 | 2500 | 150 |
| octyl acetate/octil acetato | 16000 | 28300 | 42000 | 13000 |
| benzyl benzoate/benzil benzoaoto | 280 | 950 | 2000 | 180 |
| Cembrene/unvaried | 200 | 870 | 1870 | 190 |
| n-octanol/n-octanolo | 80 | 250 | 2000 | 65 |
| neryl acetate/neryl acetato | 600 | 1200 | 3000 | 500 |
| cis-retinal/cis-retinale | 200 | 550 | 2100 | 150 |
| farnesyl acetate/farnesil acetato | 100 | 300 | 2500 | 95 |
| Verbenone/unvaried | 1550 | 3500 | 6000 | 1200 |
| ursolic acid/acido ursolico | 1500 | 2500 | 5000 | 1300 |
| alpha-amyrin/alfa-amirina | 1500 | 2500 | 4000 | 1350 |
| Epilupeol/epilupeolo | 800 | 1650 | 3200 | 600 |
| Mansubiol/mansubiolo | 1300 | 2600 | 6000 | 1000 |
| Phytol/fitolo | 1200 | 2800 | 5800 | 900 |
| Aromadendrene/unvaried | 1200 | 2800 | 5600 | 1100 |
| beta-bowellic acid (BA)/acido beta- | 8000 | 18000 | 28000 | 7000 |
| boswellico | | | | |
| 3-O-acetyl-beta-boswellic | 8000 | 18000 | 28000 | 7000 |
| acid (A-beta-BA)/acido 3-O-acetil | | | | |
| beta bowellico | | | | |
| 11-keto-beta-boswellic/acido 11-keto | 6500 | 15000 | 25000 | 5000 |
| boswellico acid (KBA) | | | | |
| 3-O-acetyl-11-keto boswellic | 24300 | 38500 | 52000 | 18000 |
| acid (AKBA)/acido 3-=-11-keto | | | | |
| bosewllico | | | | |
| Incensole/incensolo | 3000 | 6000 | 9000 | 2000 |
| incensole oxide/incensolo ossido | 6000 | 9000 | 11000 | 4000 |
| incensole acetate/incensolo acetato | 4000 | 6500 | 9000 | 3000 |
| Olibanulol/olibanulolo | 1000 | 2500 | 3800 | 600 |
| otillon acetate/otillon acetato | 800 | 1400 | 2100 | 500 |
| TOTAL QUANTITIES EXTRACTED | 135720 | 236255 | 377370 | 99060 |

TABLE 9

| | S1 | S2 | S3 | S4 |
|---|---|---|---|---|
| COMPOUND | ppm | ppm | ppm | ppm |
| germacrene | 900 | 1200 | 1900 | 650 |
| furanodiene | 1150 | 2000 | 2950 | 700 |
| 2-methoxyfuranodiene/2- | 2500 | 3500 | 4100 | 1500 |
| metossifuranodiene | | | | |
| 4,5-dihydroxyfuranodiene-6-one/ | 550 | 1000 | 2200 | 400 |
| unvaried | | | | |
| beta-selinene | 300 | 550 | 1200 | 200 |
| lindestrene | 2000 | 3200 | 4500 | 1500 |
| furanoeudesma-1,3-diene/unvaried | 15750 | 21000 | 32000 | 11000 |
| beta-elemene/unvaried | 8400 | 11000 | 15000 | 6000 |
| gamma-elemene/unvaried | 2370 | 3200 | 4500 | 1800 |
| delta-elemene/unvaried | 550 | 850 | 1250 | 440 |
| Elemon/unvaried | 210 | 800 | 1250 | 150 |
| Isofuranogermacrene/unvaried | 3385 | 4500 | 5500 | 2500 |
| Curzerenone/unvaried | 1500 | 2100 | 3500 | 1000 |
| alpha-cubebene/alfa cubebene | 1500 | 2000 | 3500 | 890 |
| beta-bourbonene/unvaried | 450 | 2000 | 3500 | 400 |
| alpha-pinene/alfa-pinene | 210 | 800 | 1250 | 200 |
| Myrcene/mircene | 150 | 280 | 380 | 120 |
| beta-ylangene/unvaried | 150 | 250 | 420 | 110 |
| alpha-gurjunene/unvaried | 500 | 800 | 1500 | 400 |
| Alloaromatoandrene/unvaried | 2500 | 3200 | 4200 | 1800 |
| Bicyclogermacrene/ | 5000 | 8000 | 11000 | 4000 |
| biciclogermacrene | | | | |
| alpha-guaiene/alfa-guaiene | 2500 | 3500 | 4200 | 2000 |
| gamma-cadinene/unvaried | 1500 | 2800 | 4300 | 1000 |
| delta-cadinene/unvaried | 800 | 1200 | 2800 | 650 |
| tau-cadinol/tau cadinolo | 1700 | 2800 | 3500 | 1200 |
| Lindestrene/unvaried | 200 | 600 | 800 | 150 |
| beta-sesquiphellandrene/beta | 150 | 300 | 550 | 100 |
| sesquifellandrene | | | | |
| 2-Omethyl.8,12 epoxygermacrene/ | 500 | 800 | 1500 | 300 |
| unvaried | | | | |
| 1,(10)4.7.11-tetraene isomer/unvaried | | | | |
| Bicyclogermancrene/ | 1800 | 2800 | 4000 | 1200 |
| biciclogermacrene | | | | |
| Myrrhone/mirrone | 300 | 600 | 800 | 150 |
| Epicurzerenone/unvaried | 2000 | 3000 | 4500 | 1500 |
| eudesm-4(15)-ene-1-beta | 850 | 1200 | 2000 | 600 |
| alpha-6-diol/unvaried | | | | |
| 4-O.methylglucoronic acid/acido 4-O | 1500 | 2800 | 3500 | 1000 |
| metilclucoronico | | | | |
| Guggulsterol/guggulsterolo | 4500 | 7000 | 9500 | 4000 |
| Quercetin/quercetina | 2500 | 4800 | 6500 | 1800 |
| quercetin-3-O-beta-D-galactoside/ | 200 | 400 | 650 | 120 |
| unvaried | | | | |
| ellargic acid/acido ellargico | 1200 | 2000 | 2500 | 800 |
| quercetin-3-O-alpha-L-arabinose/ | 1500 | 2100 | 3200 | 1100 |
| unvaried | | | | |
| Pelagornidin/pelagomidina | 1300 | 2400 | 3000 | 800 |
| linoleic acid/acido linoleico | 600 | 800 | 1800 | 500 |
| alpha-mirrholic acid/acido | 5700 | 9000 | 12000 | 4000 |
| alfa-mirrolico | | | | |
| beta-mirrholic acid/acido | 5300 | 8200 | 12000 | 4100 |
| beta-mirrolico | | | | |
| gamma-mirrholic acid/acido gamma | 4600 | 7800 | 9500 | 3800 |
| mirrolico | | | | |
| Guggulsterone/unvaried | 7800 | 8000 | 9800 | 6000 |
| bornyl acetate/bornyl acetato | 120 | 200 | 350 | 80 |
| d-limonene/unvaried | 200 | 400 | 650 | 100 |
| Linalool/linaloolo | 80 | 400 | 650 | 30 |
| Methylclavicol/metilclavicol | 150 | 200 | 350 | 95 |
| alpha-terpineol/alfa terpineolo | 250 | 300 | 420 | 180 |
| dammarane triterpene/unvaried | 1275 | 1850 | 2500 | 800 |
| TOTAL QUANTITIES | 101098 | 164880 | 209920 | 73915 |
| EXTRACTED | | | | |

TABLE 10

| | S1 | S2 | S3 | S4 |
|---|---|---|---|---|
| COMPOUND | ppm | ppm | ppm | ppm |
| Benzoic acid/acido benzoico | 2000 | 3000 | 4200 | 1600 |
| dihydrocinnamicacid/acido | 500 | 600 | 800 | 450 |
| diidrocinnamico | | | | |
| Z-cinnamic acid/acido Z-cinnamico | 20000 | 26000 | 35000 | 10000 |
| 3-phenyl-3-hydroxypropanoic acido | 5000 | 8000 | 11000 | 4000 |
| methoxyphenylpropanoic acido | 750 | 900 | 1400 | 690 |
| 4-hydroxybenzoic acid/acido | | | | |
| 4-idrossibenzoico | 750 | 900 | 1450 | 550 |
| Z-P-coumaric acid/ | 21 | 60 | 110 | 18 |
| acido Z-P-coumarico | | | | |
| E-p-coumaric acid/ | 4300 | 6500 | 9500 | 3800 |
| acido E-p.coumarico | | | | |
| ferulic acid/acido ferulico | 1200 | 1800 | 2100 | 900 |
| caffeic acid/acido caffeico | 20 | 60 | 110 | 15 |
| benzyl alchool/alcool benzilico | 125 | 210 | 315 | 90 |
| Hydroquinone/idrochinone | 750 | 1200 | 1950 | 600 |
| 4-hydroxybenzylaldheide/4- | 350 | 620 | 950 | 260 |
| idrossibenzaldeide | | | | |
| cinnamic alcohol/alcool cinnamico | 500 | 900 | 1500 | 400 |
| hydroxy acetophenone/ | 30000 | 35000 | 46000 | 25000 |
| idrossiiacetofenone | | | | |
| oleic acid/acido oleico | 20 | 60 | 120 | 20 |
| stearic acid/acido stearico | 370 | 600 | 1250 | 280 |
| palmitic acid/acido palmitico | 20 | 40 | 80 | 20 |
| benzyl benzoate/benzil benzoato | 4500 | 6000 | 8500 | 3500 |
| benzyl methoxybenzoate/ | 8500 | 12000 | 18000 | 7000 |
| benzilmetossibenzoate | | | | |
| benzyl-Z-coumarate/ | 250 | 400 | 800 | 150 |
| benzil-z-coumarato | | | | |
| benzyl ferulate/benzilferulato | 1500 | 2000 | 2900 | 1200 |
| benzyl caffeate/benzilceffeato | 1500 | 2000 | 2900 | 1000 |
| phenetyl caffeate/fenetilcaffeato | 1800 | 2500 | 3200 | 1200 |
| cynnamil caffeate/cinnamilcaffeato | 1200 | 2600 | 3000 | 800 |
| pinostrobin chalcone/unvaried | 350 | 450 | 800 | 250 |
| Pinocembrin/pinocembrina | 2000 | 2500 | 4000 | 1300 |
| Pinobanksin/pinobanksina | 250 | 350 | 600 | 140 |
| Sakuratenin/unvaried | 20 | 80 | 120 | 20 |
| Galangin/galangina | 1800 | 2800 | 4200 | 1300 |
| quercetinquercetina | 1500 | 2200 | 3800 | 1000 |
| pinobaksin-3-O-acetate/ | 600 | 800 | 1600 | 450 |
| pinobaksin-3-O-acetato | | | | |
| Glicerol/gliceolo | 150 | 200 | 400 | 120 |
| Kaempferol/kaempferolo | 8000 | 9000 | 12000 | 6000 |
| Farnesol/farnesolo | 4500 | 5200 | 6000 | 3500 |
| Apigenin/apigenina | 1500 | 2000 | 3000 | 1200 |
| Alpinone/unvaried | 1500 | 1900 | 2500 | 1100 |
| Morin/morina | 1500 | 1900 | 2500 | 1400 |
| 2′-6′-dihydroxy-4′-methoxy | 50 | 90 | 150 | 20 |
| Dihydrochalcone/unvaried | | | | |
| 2′,4′.6′-trihysroxyhydrochalcone/ | 4000 | 6000 | 8000 | 3000 |
| unvaried | | | | |
| Isokuranetin/unvaried | 2000 | 2500 | 3500 | 1500 |
| butyl caffeate/butilcaffeato | 1500 | 2700 | 3500 | 1000 |
| miristic acid/acido miristico | 1500 | 2700 | 3500 | 950 |
| beta-phenyl-ethyl-caffeate/ | 1500 | 2700 | 3500 | 900 |
| beta fenil-etil-caffeato | | | | |
| TOTAL COMPOUNDS | 125000 | 165840 | 221005 | 89593 |
| EXTRACTED | | | | |

1. A method of extracting active molecules from a vegetable substrate, said method comprising contacting said substrate with an extraction liquid, said extraction liquid comprising:
an extraction gas in the gaseous state at a temperature of 23° C. and pressure of 1 atmosphere, and
an extraction solvent comprising acetic acid.

2. The method according to claim 1, wherein the active molecules extracted from the vegetable substrate are selected from the group comprising terpenes, flavonoids, anthocyans and catechins.

3. The method according to claim 1, wherein the vegetable substrate is selected from the group comprising natural resins, fossil resins, seed, barks, leaves, algae, essential oils, roots, vegetables and fruit.

4. The method according to claim 1, wherein said extraction gas is selected from the group comprising helium, neon, argon, krypton, xenon, carbon dioxide, nitrogen, oxygen or mixtures thereof.

5. The method according to claim 4, wherein said extraction gas is selected from the group comprising argon, nitrogen, carbon dioxide, an argon and nitrogen mixture, an argon and carbon dioxide mixture, a nitrogen and carbon dioxide mixture or a mixture of argon, nitrogen and carbon dioxide.

6. The method according to claim 1, wherein said extraction solvent comprises acetic acid, in an amount from 99 to 5% by weight, relative to the total weight of said solvent, and water in an amount from 1 to 95% by weight, relative to the total weight of said solvent.

7. The extraction method according to claim 1, wherein the extraction of active molecules that is obtained when said substrate is placed in contact with an extraction liquid is conducted at an extraction temperature from 20 to 90° C., at a pressure from 1 to 5 atmospheres and for an extraction time from 1 to 8 hours.

8. The method according to claim 1, wherein said extraction liquid comprises:
said extraction gas, which is selected from the group comprising argon, nitrogen, carbon dioxide, an argon and nitrogen mixture, an argon and carbon dioxide mixture or a nitrogen and carbon dioxide mixture, and
said extraction solvent, which is a solution of acetic acid in water.

9. An extract obtainable with the extraction method according to claim 1, wherein said extract contains the active molecules extracted from said vegetable substrate, and said molecules belong to the group comprising terpenes, flavonoids, anthocyanins and catechins, in free form.

10. A food composition, a supplement product, a nutraceutic composition or a pharmaceutical product for external or internal use, comprising the extract of claim 9.

11. The method according to claim 2, wherein said terpenes are selected from the group comprising hemiterpenes, monoterpenes, sesquiterpenes, diterpenes, sesterpenes, triterpenes and tetraterpenes.

12. The method according to claim 2, wherein said flavonoids are selected from the group comprising flavones, isoflavones and neoflavones.

13. The method according to claim 2, wherein said anthocyans are selected from the group comprising delphinidin, petunidin, cyanidin, anthocyanin, malvidin, peonidin, tricetinidin, apigeninidin, pelargonidin and proanthocyanin; and

14. The method according to claim 2, wherein said catechins are selected from the group comprising polyphenolic compounds selected from among: epigallocatechin-3-gallate (EGCG), epigallocatechin (EGC), epicatechin-3-gallate (ECG), epicatechin (EC), gallocatechin and catechin.

15. The method according to claim 4, wherein said extraction gas is added to said extraction solvent in a liquid state at a concentration of from 0.1 to 10% by volume, relative to 100 parts by weight of extraction solvent.

16. The method according to claim 6, wherein said extraction solvent comprises acetic acid in an amount from 80 to 40% by weight, relative to the total weight of said solvent and water in an amount from 20 to 60% by weight, relative to the total weight of said solvent.

17. The method according to claim 8, wherein said gases are present in the extraction gas in a ratio comprised from 1:3 to 3:1.

18. The method according to claim 8, wherein said solution comprises acetic acid in an amount of 10 to 95% by weight and water in an amount of 90 to 5% by weight, relative to the total weight of said solvent.

19. The method according to claim 18, wherein said solution comprises acetic acid in an amount of 80 to 40% by weight acid and water in an amount of 20 to 60% by weight, relative to the total weight of said solvent.

20. The food composition, the supplement product, the nutraceutic composition or the pharmaceutical product according to claim 10 for topical or oral administration.
